(19) United States
(12) Patent Application Publication
(10) Pub. No.: US 2007/0066659 A1
(43) Pub. Date: Mar. 22, 2007

(54) Use of tenatoprazole for the treatment of gastroesophageal reflux disease

(76) Inventor: Francois Schutze, (US)

Correspondence Address: BUCHANAN, INGERSOLL & ROONEY PC, POST OFFICE BOX 1404, ALEXANDRIA, VA, 22313-1404, US

(21) Appl. No.: 10/531,900

(22) PCT Filed: Oct. 21, 2003
(86) PCT No.: PCT/FR03/03122

§ 371 Date: Jun. 23, 2006

(30) Foreign Application Priority Data
Oct. 21, 2002 (FR) 021311365

(51) Int. Cl. A61K 31/4439

(52) U.S. Cl. 514/338

(57) ABSTRACT

The invention relates to a novel therapeutic application of tenatoprazole. Tenatoprazole and the salts thereof can be used in the production of a medicament for the treatment of atypical and esophageal symptoms of gastroesophageal reflux, gastrorrhagia and dyspepsia.

The present invention concerns the treatment of diseases related to gastroesophageal reflux, digestive bleeding and dyspepsia, and more particularly the use of tenatoprazole in the manufacture of a medicament intended for the treatment of diseases related to gastroesophageal reflux, digestive bleeding and dyspepsia.

Tenatoprazole, or 5-methoxy-2-[[(4-methoxy-3,5-dimethyl-2-pyridyl)methyl]sulfinyl]imidazo[4,5-b]pyridine, is described in Patent No. EP 254588. It belongs to the group of drugs considered as proton pump inhibitors, which are useful in the treatment of gastric and duodenal ulcers. Other proton pump inhibitors include omeprazole, rabeprazole, pantoprazole, and lansoprazole, which all exhibit structural analogy and belong to the group of pyridinyl-methyl-sulfinyl-benzimidazoles. These compounds are sulfoxides exhibiting asymmetry at the level of the sulphur atom, and are therefore generally seen in the form of a racemic mixture of two enantiomers.

The first known derivative in this series was omeprazole, described in Patent No. EP 005.129 and endowed with properties which inhibit the secretion of gastric acid, and which is widely employed as an anti-ulcerative agent in human therapeutics.

Omeprazole has also been envisaged for the treatment of gastroesophageal reflux disorders, but its activity in this indication is not totally satisfactory. Indeed, studies have shown that its duration of action, as in the case with other proton pump inhibitors, is insufficient to treat nocturnal reflux effectively.

Gastroesophageal reflux is thought to be mainly due to a disorder of motility, characterised by abnormally frequent, transient relaxation and a loss of sphincter tone in the lower oesophagus. The effect of these abnormalities is to allow reflux of the stomach contents into the oesophagus. Furthermore, in patients suffering from gastroesophageal reflux, the elimination of reflux acidity is usually about 50% slower than in normal subjects, and the resistance of the oesophageal wall against acid attack is markedly diminished. Thus, acid secretion by the stomach plays an important role in the onset and persistence of oesophageal mucosal lesions in patients suffering from gastroesophageal reflux.

Various studies have shown that the severity of symptoms in patients suffering from gastroesophageal reflux is proportional to the duration of oesophageal mucosal exposure to acid (Howden C W, Burget D W, Hunt R H “Appropriate acid suppression for optimal healing of duodenal ulcer and gastrooesophageal reflux disease”, Scand. J. Gastroenterol, Suppl (1994) 201:79-82). Thus, non-symptomatic subjects have an exposure of about 1% (percentage of duration of exposure to acid during a day), while those who are affected occasionally by gastroesophageal reflux have a rate of exposure close to 2%, subjects with daily symptoms a rate of 3% and those presenting with endoscopic lesions a rate ranging from 6% to 12%, depending on the severity of the lesion. These studies were conducted in patients with exposure to acid at a pH lower than 4, i.e. abnormally low in the oesophagus, where normal values are usually between 5 and 7.

The studies thus demonstrated that the longer the exposure to acid, the more the symptoms and oesophageal mucosal lesions were severe.

In addition, studies have shown that the suppression of acid resulting from appropriate drug therapy is correlated with the rate of lesion recovery, the important parameters being the duration of acid inhibition and its amplitude. For this reason, patients suffering from gastroesophageal reflux are often prescribed antacids, histamine receptor antagonists or proton pump inhibitors, the aim being to relieve their symptoms. However, most of the medicaments used are not fully satisfactory as they only procure partial relief from symptoms, or their duration of action is too short, thus requiring repeated intakes of medication.

Similarly, in the treatment of dyspepsia, studies have shown that proton pump inhibitors can provide a certain degree of relief, but few treatments are effective.

Functional dyspepsia is made up of a series of changing symptoms linked to the diet and, at varying degrees, associating pain or discomfort in the upper abdomen, a sensation of early satiety or slow digestion, nausea, vomiting, etc. The pathophysiology of functional dyspepsia still remains poorly understood.

It has been shown that in certain patients, particularly those suffering from functional dyspepsia of a pseudo-ulcer type or mimicking the symptoms of gastroesophageal reflux, relief can be obtained by the administration of a medicament from the therapeutic category of proton pump inhibitors, such as omeprazole or lansoprazole. During these studies, therapeutic benefits were mainly observed in subjects presenting with increased exposure to oesophageal acid. However, the relatively short elimination half-life of proton pump inhibitors constitutes a problem with regards the suppression of gastric acidity, so that they cannot be prescribed to ensure the effective relief of functional dyspepsia.

Several techniques have been developed to provide formulations containing proton pump inhibitors likely to have improved properties. For example, WO 02.072070 discloses microparticles obtained by a spray-freezing technique, with a high content of omeprazole or esomeprazole magnesium, which can be coated with an enteric coating to protect them from contact with the acidic gastric juice. WO 99.59544 describes orally disintegrable tablets containing fine granules comprising a composition coated with an enteric coating layer. Said composition comprises an acid labile active substance such as lansoprazole. The microparticles and tablets containing fine granules described in said patents are supposed to be useful in the usual treatments of gastric acid related diseases, but no clinical result is given. Adis R&D Profile (2002:3(4) 276-277) relates to some properties of tenatoprazole and mentions that it is registered in Japan for reflux oesophagitis in April 2002, but this information is false since such registration was never obtained because this possible use of tenatoprazole was not demonstrated by then.

For these reasons, there is still a need today for a medicament which can effectively treat and relieve the symptoms of patients suffering from gastroesophageal reflux and dyspepsia.

Studies and experimental work carried out by the applicant have shown unexpectedly that tenatoprazole can be efficiently used to treat diseases related to gastroesophageal reflux and dyspepsia, while omeprazole and other proton pump inhibitors with an analogous structure do not procure any satisfactory treatment efficacy in these indications.

The aim of the present invention is therefore the use of tenatoprazole in the treatment of atypical and oesophageal symptoms of gastroesophageal reflux, digestive bleeding and dyspepsia, and the use of tenatoprazole in the manufacture of a medicament intended for the treatment of atypical and oesophageal symptoms of gastroesophageal reflux, digestive bleeding and dyspepsia.

Like omeprazole and other sulfoxides with an analogous structure, tenatoprazole includes an asymmetric structure and can therefore be presented in the form of a racemic mixture or of its enantiomers.

Unlike other proton pump inhibitors, such as, for example, omeprazole or esomeprazole, tenatoprazole is endowed with a markedly longer duration of action, resulting from a half-life which is some seven times longer. Thus, the medical data collected have shown that tenatoprazole ensures a degree of symptom relief and lesion healing which is superior to those seen with other medicaments belonging to the same therapeutic category of proton pump inhibitors, thus enabling its effective use in the treatment of atypical and oesophageal symptoms of gastroesophageal reflux, digestive bleeding and dyspepsia.

The present invention allows for the use of tenatoprazole to provide a greater degree of relief from the atypical symptoms of gastroesophageal reflux, and more particularly nocturnal, atypical symptoms which today remain refractory to treatment with standard proton pump inhibitors, such as omeprazole. Similarly, the present invention provides a marked advantage in the occasional treatment of atypical symptoms of gastroesophageal reflux, where the volume of drug intake is conditional on the duration of the therapeutic effect.

Another advantage of the present invention is that tenatoprazole can also act effectively on Barrett's oesophagus, or endobrachyoesophagus, which is defined by the presence of an intestinal-type mucosa (cylindrical) at the level of the lower oesophagus or the gastroesophageal junction. This condition is a complication of peptic oesophagitis, and can in certain cases degenerate into an adenocarcinoma.

Patients suffering from Barrett's oesophagus usually experience more serious than average gastroesophageal reflux, and the degree of acidity of the reflux may have harmful consequences on cell differentiation and proliferation, favouring the development of dysplasia. It is therefore important to be able to reduce acid secretion in patients presenting with symptoms related to gastroesophageal reflux and histological lesions related to Barrett's oesophagus.

Treatment must procure the maximum suppression of gastroesophageal reflux acidity in the case of Barrett's oesophagus, and the administration of tenatoprazole indeed enables this, and more particularly prevents attacks of nocturnal heartburn, which is not achieved by the medicaments currently available, even standard proton pump inhibitors.

As shown below, tenatoprazole can be distinguished from other proton pump inhibitors because of its astonishingly longer elimination half-life, and also its considerable degree of tissue exposure, as has been demonstrated during experiments conducted by the applicant.

A phase I study in Caucasian individuals (n=8 per group) made it possible to demonstrate the influence of different doses of tenatoprazole on pharmacokinetic parameters, in the case of the oral administration of a single dose and a daily dose for a period of 7 days.

The doses tested were 10, 20, 40 and 80 mg of tenatoprazole.

The results obtained are grouped in Table 1 below.

TABLE 1

| | Single dose | | | | Repeated dose (7 days) | | | |
|---|---|---|---|---|---|---|---|---|
| | 10 mg | 20 mg | 40 mg | 80 mg | 10 mg | 20 mg | 40 mg | 80 mg |
| Cmax (μg/ml) | 0.9 | 2.4 | 5.3 | 8.3 | 1.6 | 3 | 5.5 | 11.8 |
| Tmax (h) | 4 | 4 | 3 | 3 | 3 | 2 | 3 | 2 |
| T½ (h) | 5 | 6 | 6 | 7 | 5 | 8 | 9 | 9.2 |
| AUC 0-t | 8 | 24 | 43 | 97 | 13 | 36 | 75 | 218 |

In this table, the abbreviations employed have the following meaning:

Cmax maximum concentration

Tmax time required to attain maximum concentration

T½ elimination half-life AUC_{0-T} area under the curve, between time 0 and the last measurable concentration.

The results shown in Table 1 above demonstrate that the mean elimination half-lives were between 5 and 6 hours after the administration of a single dose, and between 5 and 9.5 hours after administration for seven days, depending on the dose. Tenatoprazole also exhibited high AUC values (area under the curve), providing evidence of a low rate of metabolism and/or high bioavailability via the oral route. Furthermore, whatever the conditions of administration, single or repeated, the Cmax, AUC₀₋ₜ and AUC_{0-inf} values increased in a linear fashion. The AUC_{0-inf} value was calculated by extrapolation.

A comparison of AUC values between two proton pump inhibitors, lansoprazole and omeprazole, was made by Tolman et al. (J. Clin. Gastroenterol., 24(2), 65-70, 1997), but this did not enable a judgement as to the superiority of one product over the other. Indeed, different criteria must be taken into account, i.e. the time required for pump regeneration, and the period above the minimum concentration necessary to inhibit proton pumps. With respect to the pump regeneration time, it is observed that pumps usually have a half-life of about 30 to 48 hours, and are therefore totally renewed every 72 to 96 hours.

Thanks to the pharmacokinetic properties described above, tenatoprazole can counteract the proton pump regeneration phenomenon by maintaining an inhibitory concentration for a sufficiently long period of time to meet the two criteria specified previously.

Thus, the prolonged exposure (determined by the AUC), bound to the long half-life of tenatoprazole, endow it with a longer presence at the sites of activity and thus procure a pharmacodynamic effect which is prolonged over time. Experiments have thus shown that tenatoprazole is endowed with a plasma half-life/pump regeneration time ratio which is notably higher than that seen with other proton pump inhibitors, thus permitting its use to treat diseases for which the treatments currently available are of poor efficacy, in particular atypical and oesophageal symptoms of gastroesophageal reflux, dyspepsia and Barrett's oesophagus.

More particularly, according to the present invention, tenatoprazole can be used to treat atypical symptoms of gastroesophageal reflux such as asthma and dyspnoea attacks of an asthmatic type, pharyngitis, dysphonia, pseudo-angina, paroxysmal cough and nocturnal cough. It is also particularly effective in treating pseudo-ulcer dyspepsia. And, as shown above, it can also be used successfully to treat Barrett's oesophagus.

In the treatment of atypical and oesophageal symptoms of gastroesophageal reflux, digestive bleeding, particularly due to ulcers, and dyspepsia, tenatoprazole can be administered in the usual forms adapted to the mode of administration chosen, for example via the oral or parenteral routes, but preferably via the oral or intravenous routes. For example, it is possible to employ tablet or capsule formulations containing tenatoprazole as the active substance, or drinkable solutions or emulsions or solutions for parenteral use containing a tenatoprazole salt with a standard, pharmaceutically acceptable substrate. The tenatoprazole salt can be selected from sodium, potassium, magnesium or calcium salts.

As an example, an appropriate formulation for tablets containing 20 mg of tenatoprazolecombined with pharmaceutically acceptable supports and excipients is shown herewith:

| | | |
|---|---|---|
| tenatoprazole | 20.0 | mg |
| lactose | 32.0 | mg |
| aluminium hydroxide | 17.5 | mg |
| hydroxypropylcellulose | 12.1 | mg |
| talc | 4.5 | mg |
| titanium dioxide | 3.2 | mg |
| magnesium stearate | 1.0 | mg |
| usual excipients | q.s.p. 160 | mg |

Dosage is determined by the practitioner as a function of the patient's state and the severity of the disorder. It is generally between 10 and 120 mg, and preferably between 20 and 40 mg of tenatoprazole per day, corresponding for example to one intake per day of 1 to 2 tablets, each containing 20 or 40 mg of the active substance, for a period of time which may be between 4 and 12 weeks, in the event of initial or maintenance therapy. In the case of a paediatric formulation, adapted to young children, for example a drinkable solution, the unit dose may be lower, for example two or five mg. In the case of severe disorders, it may be effective to administer the medicine initially via the intravenous route, and subsequently via the oral route. Furthermore, the invention has the advantage of allowing effective, sequential treatment through the simple administration of a single tablet per week, containing 20 or 40 mg.

Some clinical examples are given below, which show the effects of treatment on patients suffering from gastroesophageal reflux or dyspepsia, treated by the oral administration of tenatoprazole.

TABLE 2
Treatment of patients with gastroesophageal reflux symptoms

| | Predominant | Duration of | | Evolution of | |
|---|---|---|---|---|---|
| Age/Gender | symptom | treatment | | symptom | Safety |
| 47/F | n.h. | 8 | weeks | ++ | +++ |
| 38/M | h. | 8 | weeks | +++ | +++ |
| 35/F | n.h. | 4 | weeks | ++ | +++ |
| 34/M | h. | 8 | weeks | +++ | +++ |
| 45/M | n.h. | 8 | weeks | +++ | +++ |
| 30/M | n.h. | 8 | weeks | +++ | ++ |
| 49/F | r. | 8 | weeks | ++ | +++ |
| 42/M | h. | 8 | weeks | ++ | +++ |
| 38/F | n.h. | 8 | weeks | +++ | +++ |
| 25/F | h. | 12 | weeks | +++ | +++ |
| 28/M | n.h. | 4 | weeks | +++ | +++ |
| 39/F | n.h. | 4 | weeks | + | +++ |
| 41/M | h. | 8 | weeks | +++ | +++ |
| 36/F | r. | 8 | weeks | +++ | ++ |

h.: heartburn
n.h.: nocturnal heartburn
r. regurgitation
The symbols + to +++ indicate the evolution of symptoms and safety, ranging from moderate to highly favourable.

Treatment consisted in the daily administration of a tablet containing 20 mg tenatoprazole. The table shows that treatment was perfectly tolerated in 12 out of 14 cases, and well tolerated in the other two patients, while the evolution observed in symptoms was generally very favourable.

TABLE 3
Treatment of patients with atypical symptoms of gastroesophageal reflux

| | Predominant | link with | Duration of | | Evolution of | |
|---|---|---|---|---|---|---|
| Age/Gender | symptom | GERD | treatment | | symptom | Safety |
| 44/M | pharyngitis | + | 4 | weeks | ++ | +++ |
| 36/M | dysphonia | ++ | 5 | weeks | +++ | +++ |
| 34/F | dysphonia | ++ | 4 | weeks | ++ | +++ |
| 45/M | pseudo-angina | ++ | 8 | weeks | +++ | +++ |
| 29/F | noct. cough | +++ | 7 | weeks | +++ | +++ |
| 27/M | dental caries | + | 12 | weeks | 0 | ++ |
| 33/M | asthma | ++ | 12 | weeks | ++ | +++ |
| 34/F | pharyngitis | ++ | 8 | weeks | ++ | +++ |
| 36/F | noct. cough | ++ | 8 | weeks | +++ | ++ |
| 26/M | asthma | ++ | 12 | weeks | +++ | +++ |
| 49/M | pseudo-angina | ++ | 12 | weeks | +++ | +++ |
| 31/F | pharyngitis | + | 8 | weeks | + | +++ |

GERD: gastroesophageal reflux disease.

The results in the table above show that the evolution of symptoms was particularly favourable in cases where a link with gastroesophageal reflux was the most clear.

TABLE 4
Treatment of patients with symptoms of functional dyspepsia

| | Predominant | Duration of | | Evolution of | |
|---|---|---|---|---|---|
| Age/Gender | symptom | treatment | | symptom | Safety |
| 47/F | n. | 4 | weeks | ++ | +++ |
| 38/M | g.f. | 8 | weeks | +++ | +++ |
| 35/F | h. | 8 | weeks | +++ | +++ |
| 34/F | e.s. | 8 | weeks | +++ | +++ |
| 45/M | e.p. | 6 | weeks | +++ | ++ |
| 30/F | n.h. | 8 | weeks | +++ | +++ |
| 49/F | n. | 8 | weeks | ++ | +++ |
| 42/M | e.s. | 6 | weeks | ++ | +++ |
| 38/F | e.p. | 8 | weeks | +++ | ++ |
| 25/F | e.d. | 12 | weeks | ++ | +++ |
| 28/M | d. | 4 | weeks | + | +++ |
| 39/F | e.p. | 4 | weeks | ++ | ++ |
| 41/M | h. | 6 | weeks | +++ | +++ |
| 36/F | e.d. | 8 | weeks | +++ | ++ |
| 44/F | n. | 10 | weeks | +++ | +++ |

h.: heartburn
n.h.: nocturnal heartburn
n.: nausea
g.f.: sensation of gastric fullness
e.s.: early satiety
e.p.: epigastric pain
e.d.: epigastric discomfort
d.: distension

These results confirm the efficacy of tenatoprazole, administered according to the invention, in the treatment of dyspepsia.

Two open-labeled studies have been conducted in order to evaluate the efficacy and safety of tenatoprazole for gastroesophageal reflux disease. 22 patients in the first study and 24 patients in the second study, of more than 20 years of age, suffering from erosive and/or ulcerative type reflux esophagitis (diagnosed by endoscopy), received tablets containing enteric-coated granules with 10 mg of tenatoprazole.

The tablets were administered orally once a day after breakfast, for a treatment period of 8 weeks, which was continued up to 12 weeks in some cases. Healing was controlled by endoscopic examination 4 weeks and 8 weeks after the first administration, or at the withdrawal from the study, the disease stages were evaluated according to the Savary and Miller classification and the treatment was stopped when healing was confirmed. The condition was evaluated as healed when disapearance of erosion was confirmed.

The endoscopic improvement rating was evaluated according to the following 6 grades: “healed”, “markedly decreased”, “moderately decreased”, “slightly decreased”, “not changed” and “aggravated”.

The improvement rating of subjective and objective symptoms, compared with the observations at the start of the study, was evaluated according to the following 6 grades: “markedly improved”, moderately improved”, “slightly improved”, “not changed”, “aggravated” and “no symptoms from the start of study”.

Healing was observed at 4 weeks and administration of tenatoprazole was discontinued at this stage for 20 cases in the first study and 23 cases in the second study. Only one patient was not healed after 8 weeks treatment.

The results are listed in Tables 5 and 6 hereinafter.

TABLE 5
Endoscopic improvement rating

| | | | | | | |
|---|---|---|---|---|---|---|
| A | B | C | D | E | F | Total |

1^{st} study

| | | | | | | | | |
|---|---|---|---|---|---|---|---|---|
| 4 | weeks | 20 | 1 | 0 | 0 | 1 | 0 | 22 |
| 8 | weeks | 21 | 0 | 0 | 0 | 1 | 0 | 22 |
| | final | 21 | 0 | 0 | 0 | 1 | 0 | 22 |

2^{nd} study

| | | | | | | | | |
|---|---|---|---|---|---|---|---|---|
| 4 | weeks | 23 | 2 | 0 | 0 | 0 | 0 | 25 |
| 8 | weeks | 24 | 0 | 0 | 0 | 0 | 0 | 24 |
| 12 | weeks | 24 | 0 | 0 | 0 | 0 | 0 | 24 |
| | final | 24 | 0 | 0 | 0 | 0 | 0 | 24 |

A: healed
B: markedly decreased
C: moderately decreased
D: slightly decreased
E: not changed
F: aggravated

TABLE 6
Improvement rating of symptoms

| | | | | | |
|---|---|---|---|---|---|
| A | B | C | D | E | Total |

1^{st} study

| | | | | | | | |
|---|---|---|---|---|---|---|---|
| 1 | week | 16 | 6 | 0 | 3 | 0 | 25 |
| 2 | weeks | 21 | 1 | 0 | 1 | 0 | 23 |
| 4 | weeks | 23 | 0 | 1 | 0 | 0 | 24 |
| 6 | weeks | 10 | 0 | 1 | 0 | 0 | 11 |
| 8 | weeks | 9 | 0 | 1 | 0 | 0 | 10 |
| 10 | weeks | 2 | 0 | 0 | 0 | 0 | 2 |
| | Final | 21 | 0 | 1 | 0 | 0 | 22 |

2^{nd} study

| | | | | | | | |
|---|---|---|---|---|---|---|---|
| 1 | week | 19 | 2 | 1 | 1 | 0 | 23 |
| 2 | weeks | 21 | 1 | 1 | 0 | 0 | 23 |
| 4 | weeks | 20 | 1 | 1 | 0 | 0 | 22 |
| 6 | weeks | 10 | 0 | 1 | 0 | 0 | 11 |
| 8 | weeks | 10 | 1 | 0 | 0 | 0 | 11 |
| 10 | weeks | 0 | 1 | 0 | 0 | 0 | 1 |
| 12 | weeks | 0 | 1 | 0 | 0 | 0 | 1 |
| | Final | 19 | 2 | 0 | 0 | 0 | 21 |

A: markedly improved
B: moderately improved
C: slightly improved
D: not changed
E: aggravated

The above results demonstrate that tenatoprazole according to the present invention is very effective in the treatment of gastroesophageal reflux disease, since healing is obtained with a 4 week treatment, to be compared with the unsatisfactory results obtained by a 8 week treatment with usual proton pump inhibitors.

1. A medicament useful in the treatment of atypical and oesophageal symptoms of gastroesophageal reflux, digestive bleeding and dyspepsia, comprised of tenatoprazole.

2-10. (canceled)

11. The medicament of claim 1, wherein said tenatoprazole is present in a dosage form containing 20 to 40 mg of active substance.

12. A method for the treatment of atypical and oesophageal symptoms of gastroesophageal reflux, digestive bleeding and dyspepsia, which comprises administering a medicament comprising tenatoprazole.

13. The method of claim 12, wherein the medicament comprising tenatoprazole is administered via the oral route.

14. Use according to claim 12, wherein the medicament comprising tenatoprazole is administered via the parental route.

15. The method of claim 12, wherein the medicament comprising tenatoprazole is administered at a rate of 10 to 120 mg per day.

16. The method of claim 15, wherein the medicament comprising tenatoprazole is administered in units containing tenatoprazole in dosage forms containing 20 to 40 mg of active substance, associated with one or several pharmaceutically acceptable excipients and substrates.

17. The method of claim 12, wherein the medicament is administered to treat the symptoms of Barrett's oesophagus.

18. The method of claim 12, wherein the medicament is administered for the treatment of nocturnal reflux.

19. The method of claim 12, wherein the medicament is administered for the treatment of pseudo-ulcer dyspepsia.

20. The method of claim 12, wherein the medicament is administered for the treatment of asthma, asthma-like acute dyspnoea, pharyngitis, dysphonia, pseudo-angina, paroxysmal cough and/or nocturnal cough.

21. The method of claim 12, wherein the medicament is administered as a salt of sodium, potassium, magnesium or calcium.
